(19) United States
(12) Patent Application Publication
(10) Pub. No.: US 2026/0061060 A1
(43) Pub. Date: Mar. 5, 2026

(54) DELIVERY COMPOSITION AND METHODS OF MANUFACTURE AND USE THEREOF

(71) Applicant: LIFT HYDRATION LLC, Lodi, NJ (US)

(72) Inventors: Andrew KNOX, Palm Springs, CA (US); Michael Mikhail, Wayne, NJ (US); James Mikhail, Fairlawn, NJ (US); Scott Knox, Cutler Bay, FL (US)

(21) Appl. No.: 19/312,792

(22) Filed: Aug. 28, 2025

Related U.S. Application Data
(60) Provisional application No. 63/688,426, filed on Aug. 29, 2024.

(51) Int. Cl. A61K 47/36; A23L 2/52; A61K 9/00; A61K 31/198; A61K 31/375; A61K 31/4415; A61K 31/455; A61K 31/525; A61K 31/714; A61K 36/481

(52) U.S. Cl. CPC A61K 47/36; A23L 2/52; A61K 9/0095; A61K 31/198; A61K 31/375; A61K 31/4415; A61K 31/455; A61K 31/525; A61K 31/714; A61K 36/481

(57) ABSTRACT

A delivery composition containing a resistant starch such as potato, sweet potato, white or brown rice, pasta, oats, or a legume such as beans or lentils, amino acids, vitamins, and additional components is described. Products such as hydration drinks containing the delivery composition are also provided.

### CROSS-REFERENCE TO RELATED APPLICATION

This application claims priority to co-pending U.S. provisional application No. 63/688,426, filed Aug. 29, 2024, the disclosure of which is herein incorporated by reference in its entirety.

### BACKGROUND OF THE INVENTION

When the human body becomes dehydrated, it has trouble balancing water and other nutrients that are used to function properly. As such, when the body is dehydrated, it is desirable to rehydrate the body as fast as possible and then maintain the hydration. Methods and formulations for hydrating the human body have been increasingly developed over the past couple of years. As these formulations have been developed, new improvements are continually desired.

### SUMMARY OF THE INVENTION

In one embodiment, aspects of the disclosure relate to a delivery composition comprising at least one carrier, wherein the at least one carrier comprises a resistant starch selected from the group consisting of potato, sweet potato, rice, oats, pasta, and a lentil.

Advantageous refinements of the invention, which can be implemented alone or in combination, are specified in the dependent claims.

In summary, the following embodiments are proposed as particularly preferred in the scope of the present invention:

### Embodiment 1

A delivery composition comprising at least one carrier, wherein the at least one carrier comprises a resistant starch selected from the group consisting of potato, sweet potato, rice, oats, pasta, and a lentil.

### Embodiment 2

The delivery composition according to Embodiment 1, wherein the resistant starch is potato maltodextrin.

### Embodiment 3

The delivery composition according to Embodiment 2, wherein the potato maltodextrin is potato maltodextrin DE 9.

### Embodiment 4

The delivery composition according to Embodiment 1, wherein the resistant starch is a potato starch, a sweet potato starch, a rice starch, or an oat starch.

### Embodiment 5

The delivery composition according to any of Embodiments 1-4, further comprising at least one amino acid.

### Embodiment 6

The delivery composition according to Embodiment 5, wherein the delivery composition comprises L-glycine and alanine.

### Embodiment 7

The delivery composition according to any of Embodiments 1-6, further comprising at least one vitamin.

### Embodiment 8

The delivery composition according to Embodiment 7, wherein the delivery composition comprises at least one of vitamin C, vitamin B3, vitamin B6, vitamin B2, and vitamin B12.

### Embodiment 9

The delivery composition according to any of Embodiments 1-8, further comprising at least one component selected from the group consisting of taurine and astragalus root powder.

### Embodiment 10

The delivery composition according to any of Embodiments 1-4, further comprising L-glycine, L-alanine, vitamin C, vitamin B3, vitamin B2, vitamin B6, taurine, and astragalus root powder.

### Embodiment 11

The delivery composition according to Embodiment 10, wherein the resistant starch is present in an amount of about 75 to about 85 weight %, the L-glycine is present in an amount of about 5 to about 7 weight %, the L-alanine is present in an amount of about 3 to about 4 weight %, the vitamin C is present in an amount of about 1 to about 3 weight %, the vitamin B6 is present in an amount of about 0.5 to about 2 weight %, the vitamin B3 is present in an amount of about 0.1 to about 0.7 weight %, the vitamin B2 is present in an amount of about 0.5 to about 2 weight %, the taurine is present in an amount of about 5 to about 7 weight %, and the astragalus root powder is present in an amount of about 0.5 to about 2 weight %, based on a total weight of the delivery composition being 100 percent.

### Embodiment 12

The delivery composition according to any of Embodiments 1-11, wherein the composition has an osmolality of about 20 to about 65 mOsmol/kg.

### Embodiment 13

A hydration drink comprising the delivery composition according to any of Embodiments 1-12, water, and at least one electrolyte.

### Embodiment 14

The hydration drink according to Embodiment 13, wherein the osmolality is about 48 mOsmol/kg.

### Embodiment 15

A drug product comprising the delivery composition according to any of Embodiments 1-12 and at least one active ingredient.

### DETAILED DESCRIPTION OF THE INVENTION

Aspects of the disclosure relate to a composition (formulation) which may function as a delivery system for delivery materials to the human body.

Unless defined otherwise, all technical and scientific terms used herein have the same meaning as commonly understood to one of ordinary skill in the art to which this invention pertains. Otherwise, certain terms used herein have the meanings as set forth in the specification. Any patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise stated, any numerical value is to be understood as being modified in all instances by the term “about.” Thus, a numerical value typically includes ±10% of the recited value. For example, the recitation of a temperature such as “10° C.” includes 9° C. and 11° C. As used herein, the use of a numerical range expressly includes all possible subranges, all individual numerical values within that range, including integers within such ranges and fractions of the values unless the context clearly indicates otherwise. Unless otherwise specified, all component weight percentages of the delivery composition are based on a total weight of the delivery composition being 100 percent.

For the purposes of this disclosure, the terms “composition” and “formulation” are used interchangeably, and both relate to a delivery system containing the specified components.

The following disclosure provides many different embodiments, or examples, for implementing different features of the invention. Specific examples of components and arrangements are described below to simplify the present disclosure. These are, of course, merely examples and are not intended to be limiting. The precise embodiments presented herein, however, are intended to be illustrative and are not intended to limit the ideas presented to only the embodiments discussed. For example, different deliverables and/or additives may be utilized in order to achieve different compositions and different flavors.

Aspects of the disclosure relate to a delivery system (composition or formulation) which contains a resistant carbohydrate (starch) carrier (component (a)), and preferably also contains amino acids (components (b) and (c)), vitamins (components (d), (g), (h), and (i)), and/or additional components ((e) and (f)), each of which has a specific and defined role in the composition. Each of these components will be described in more detail below. The delivery system may be combined with desired deliverables (e.g., water and electrolytes, active ingredients) to speed the absorption and transportation of such deliverables in the body. In one embodiment, the composition or formulation provides a rapid delivery effect (hydration effect) in the human body and also allows for the rapid delivery to extend for a long period of time.

In some embodiments, the composition contains the following components, which will be described in more detail below:
(a) A resistant carbohydrate carrier selected from potato maltodextrin, sweet potato, white or brown rice, pasta, oats, or a legume (bean or lentil).
(b) L-glycine
(c) L-alanine
(d) Vitamin C
(e) Taurine
(f) Astragalus root powder
(g) Vitamin B3
(h) Vitamin B2
(i) Vitamin B6
(j) Vitamin B2 and/or B12 (optional)

### Resistant Carbohydrate Carrier (Resistant Starch)

The resistant starch functions as the carrier in the composition and may be present in an amount of about 75 to about 85 weight percent, more preferably about 78 to about 82 weight percent, most preferably about 80 weight %. If the amount of resistant starch in the composition is greater than about 86%, subsequent absorption in the body may be reduced, whereas if the amount of resistant starch is less than about 75 weight percent, there may also be slower absorption.

This component is a resistant starch, i.e., one which is not digested by the body. It is termed “resistant” because it cannot be broken down by the digestive enzymes produced by the body, so it travels all the way to the large intestine where it feeds the gut biome. Resistant starch functions as a prebiotic, supporting the abundance of beneficial bacteria in the gut and may also enhance probiotic bacteria levels.

More specifically, RS3 are retrograded starches which are formed after cooking and then letting cool. Once an RS3 starch is cooked and cools it becomes a resistant starch due to the bonds tightening. As a cooked food cools, the linear chains of amylose realign and recrystallize into tighter more ordered structure. Such recrystallized starches resist digestion by the enzymes in the small intestine. Thus, instead of being broken down into glucose in the small intestine retrograded starch passes into the large intestine. In other words, since the body cannot break them down, they will make it all the way to the large intestine. Starches that can become resistant include potato, sweet potato, rice, oats, lentils, and beans.

Appropriate resistant starches for including in the compositions described herein include, without limitation, potato maltodextrin, sweet potato, white or brown rice, pasta, oats, or a legume (a bean or a lentil).

In preferred embodiments, the resistant starch is potato maltodextrin starch, sweet potato starch, rice starch, or oat starch.

If included, the potato resistant starch is potato maltodextrin, preferably DE 9. In this material, the potato starch is processed a very specific way such that the DE (Dextrose Equivalent) is preferably 9, very low, so that it will absorb into the system faster. The preferred potato starch is also manufactured using an enzymatic process which helps the product become water soluble. Preferably, the potato maltodextrin is non-GMO.

### L-Glycine and Alanine (Amino Acids)

The composition preferably contains at least two amino acids, L-glycine and L-alanine, preferably in specified amounts. The L-glycine may be present in the composition in an amount of about 5 to about 7 weight percent, more preferably about 5.5 to about 6.5 weight percent, most preferably about 6 weight %, and the alanine may be present in the composition in an amount of about 3 to about 4 weight percent, more preferably about 3.2 to about 3.8 weight percent, most preferably about 3.5 weight %.

The amino acid components each have a specific function in the compositions described herein. L-glycine promotes the health of the gut wall and mucosa, and a healthy gut helps with the speed of absorption of liquids into the cells. L-Glycine can help speed up the healing of ulcers and regulation of stomach acidity. L-alanine has been shown to help with the absorption of salt and water into the cells to rehydrate. Thus, the amino acids help speed the absorption and transportation of the desired deliverables (e.g., vitamins, amino acids and electrolytes) as well as to help slow fatigue. In particular embodiments, the amino acids may also be used to improve muscle function.

### Vitamin C (Ascorbic Acid)

Vitamin C (ascorbic acid) has been shown to help with the absorption of electrolytes into the cells of the body. The vitamin C may be present in the composition in an amount of about 1 to about 3 weight percent, more preferably about 1.5 to about 2.5 weight percent, most preferably about 2 weight %.

### Taurine

Taurine plays a direct role in helping electrolytes move into and out of cells. In terms of cell volume regulation, taurine is one of the main “osmolytes” in the body. It helps cells maintain their proper fluid balance by moving water and electrolytes across the cell membrane. With respect to electrolyte transport, taurine interacts with sodium, potassium, calcium, and magnesium channels in cell membranes, stabilizing how these electrolytes flow in and out of cells. The taurine may be present in an amount of about 5 to about 7 weight percent, more preferably about 5.5 to about 6.5 weight percent, most preferably about 6 weight %,

### Astragalus Root Powder

Astragalus root powder has been shown to boost energy and help with chronic fatigue syndrome. It contains antioxidants, which protect cells against damage and may be beneficial for protecting and supporting the immune system, preventing colds and upper respiratory infections, lowering blood pressure, treating diabetes, and protecting the liver. It has also been found to have antibacterial and anti-inflammatory properties. It may be present in an amount of about 0.5 to about 2 weight percent, more preferably about 0.8 to about 1.2 weight percent, most preferably about 1 weight %.

### B Vitamins: B1, B3, B2, B6 and B12

Vitamin B1, also known as thiamine, is necessary for proper functioning of electrolyte transport systems that help maintain fluid balance and avoid dehydration during exercise. Vitamin B3, also known as niacin, helps convert food into energy and supports the nervous system, digestive system, and skin. Vitamin B2, also known as riboflavin, helps the body better absorb electrolytes. Vitamin B6 is important for normal brain development and for keeping the nervous system and immune system healthy. People who have kidney disease or conditions that prevent the small intestine from absorbing nutrients from foods are most likely to be Vitamin B6 deficient. Vitamin B12 helps with the production of red blood cells and helps to create and regulate DNA.

Vitamin B3 may be present in an amount of about 0.1 to about 0.7 weight percent, more preferably about 0.2 to about 0.4 weight percent, most preferably about 0.35 weight %. Vitamin B2 may be present in an amount of about 0.5 to about 2 weight percent, more preferably about 0.8 to about 1.2 weight percent, most preferably about 1 weight %. Vitamin B6 may be present in an amount of about 0.5 to about 2 weight percent, more preferably about 0.8 to about 1.2 weight percent, most preferably about 1 weight %.

If present, Vitamin B1 may be present in an amount of about 0.5 to about 2 weight percent, more preferably about 0.9 to about 1.5 weight percent, most preferably about 1.2 weight %. If present, Vitamin B12 may be present in an amount of about 0.002 to about 0.004 weight percent, more preferably about 0.003 weight percent.

Other additives may be added to the composition and the delivery system in order to assist with the overall functioning of the composition. For example, in an embodiment an amino acid chelate may be added in order to assist with the chemical reactions. In a particular embodiment the amino acid chelate may be a chelate such as magnesium bisglycinate chelate that helps to bind magnesium to the amino acids, such as glycine, and may be added to the composition in an amount of 3.7 to 3.8 weight percent. However, any suitable amino acid chelate and any suitable amounts may be used.

Another additive may be a pH control additive which is used to help control the pH of the eventual solution derived from the composition. In an embodiment the pH control additive may be an additive such as trisodium citrate dihydrate, and may be added in an amount of 5 to about 6 weight percent, preferably about 5.5 weight percent. However, any suitable pH control additive and any suitable amount may be used.

The delivery system comprising the carrier and the one or more amino acids provides multiple pathways for the composition to deliver a desired component (e.g., water) to the human body as described further below. Specifically, the monosaccharides within the carrier provide a first, fast pathway for the desired component (e.g., water) while the polysaccharides provide a second, slower and longer lasting pathway for the desired component.

A preferred composition which contains potato maltodextrin as the resistant starch according to embodiments of the disclosure contains the components in the approximate weight percentages shown in Table A. However, the amounts shown in Table A are exemplary, not limiting, and the compositions according to the disclosure are not limited to the specific components or amounts shown in Table A.

TABLE A
Representative Composition

| Component | Approximate Weight %* |
|---|---|
| Non-GMO Potato Maltodextrin DE 9 | 80% |
| L-Glycine (Amino Acid) | 6% |
| L-Alanine | 3% |
| Vitamin C (Ascorbic Acid) | 2% |
| Taurine | 6% |
| Astragalus Root Powder | 1% |
| Vitamin B3 | 0.4% |
| Vitamin B2 | 1% |
| Vitamin B6 | 1% |
| Vitamin B12 | 0.003% |

*Total is greater than 100% due to rounding

### Methods of Forming Compositions

The method for forming the composition is not limited in any way, and the following method is exemplary only. The carrier component and the additional components (amino acids, vitamins, and additional components) are combined in a commercial, state of the art equipment that works to make a uniform powder mix, such as a blender. However, any suitable mixing process or equipment may be utilized.

Once mixed, the mixture may then be agglomerated in order to improve the delivery system's bulk density, flowability, dispersibility, stability, and solubility. For example, the agglomeration may be performed such that the powder particles are bound together to create a larger particle that will easily mix into a solution. In an embodiment the agglomeration may be performed using a fluid bed processor to unite the various properties into one consistent powder to enhance performance for the consumer. By spraying in a fluidized bed system, the individual powdery, non-flowing and often poorly soluble particles are converted into a free-flowing agglomerate with outstanding solubility. The method is particularly suitable as a preliminary step for superior instantized drinking powders that have little floating particulate or bottom sediment for maximum consumer benefit of the active nutrients.

In particular, the spray agglomeration moves very small, powder particles in the fluidized bed where they are sprayed with a binder solution or suspension. Liquid bridges are created that form agglomerates from the particles. Spraying continues until the desired size of the agglomerates is reached. After the residual moisture in the capillaries and on the surface has evaporated, hollow spaces are created in the granulate while the new structure is solidified throughout by the hardened binder. The lack of kinetic energy in the fluidized bed results in very porous structures with plenty of internal capillaries. Because of this porous character and large surface structure, the resulting agglomerated powder is very soluble when subsequently introduced to liquid.

Once the agglomeration is performed, the resultant powder of the delivery system may be considered a carrier ingredient appropriate for combination with deliverables, such as electrolytes (e.g., sodium, potassium, magnesium and/or calcium), active ingredients, and optional ingredients as described above. However, it is notable that all of the components of the delivery system play an active role; the delivery system contains no excipients or fillers.

In an embodiment in which the composition is desired to be used to deliver one or more active ingredients, once the composition has been formed, it may then be packaged, stored, transported, and/or distributed to the end user or may be combined with active ingredient(s) and optional ingredients prior to delivery to the end user. In a particular embodiment the packaging may include placing individual servings into individual packages, such as stick packages, or else may be placed in bulk containers (for, e.g., shipment) prior to placement within individual packages. If shipped in bulk, the bulk quantities may be shipped locally or overseas, where local packaging or combinations with local, more specific water types might be desirable.

Of course, while a specific process for mixing and preparing the composition has been presented above, this description is intended to be illustrative only and is not intended to limit the embodiments to the precise steps and the precise order described. Rather, any suitable order of mixing steps and methods may be utilized, such as mixing the carrier and other ingredients either simultaneously or sequentially. All such steps and orders are fully intended to be included within the scope of the embodiments.

### Products

Additional aspects of the disclosure relate to products containing the compositions described herein and additional components; the composition functions as a delivery system for the additional components to the human body. In one embodiment the delivery system may be utilized to deliver electrolytes and other components in order to help hydrate a human body, as described further below. In other embodiments, however, the delivery system may be used to provide any desired and suitable components to the human body.

In some embodiments the delivery system may be utilized to deliver more than a single desired deliverable. For example, in an embodiment in which the delivery system delivers one or more active ingredients to hydrate the human body, the delivery system may also be utilized to deliver one or more electrolytes. In an embodiment, the one or more electrolytes may be added to the composition in order to help regulate nerve and muscle function, hydrate the body, and balance blood acidity and pressure. In an embodiment the one or more electrolytes may comprise electrolytes such as calcium bisglycinate chelate, tripotassium citrate, combinations of these, or the like. However, any suitable electrolytes may be used.

In a particular embodiment the one or more electrolytes comprises a combination of calcium bisglycinate chelate and tripotassium citrate. In such an embodiment the calcium bisglycinate chelate within the composition may be present in an amount of 1 weight % and the tripotassium citrate may be present in an amount of 6 weight %. However, any suitable amounts may be used.

For example, one embodiment of the disclosure relates to a hydration drink containing the composition described herein, water, and electrolytes, facilitating delivery of the electrolytes and rapid hydration. A medication containing the composition described herein and an active ingredient (such as, for example and without limitation, ibuprofen, acetaminophen, naproxen, or other NSAIDS or any other drug to be contemplated) facilitates rapid delivery of the active ingredient to the body. Other products which may be contemplated which contain the composition described herein include, without limitation, a libido enhancer composition, a hangover recovery composition, an energy/focus booster composition, an immune boost composition, a stress release composition, and a weight control composition.

With respect to a hydration drink, an end user may combine a composition as described herein (which may be provided and available as a solid powder mixture) with a commercially available hydration drink or a drink prepared by the user containing desired components. Also within the scope of the disclosure is a hydration drink containing water, electrolyte, and the composition as described herein, which may be obtained or purchased in ready to drink format by the end user.

Such a hydration drink containing the delivery system (composition) as described herein provides the user with a high tonicity (e.g., a measure of osmolality in a beverage), hypotonic drink (instead of lower tonicity drinks such as isotonic drinks which have a large sugar content), wherein the hypotonic drink has zero sugar, is non-GMO, gluten free, vegan, and all natural. For example, the hypotonic drink may have an osmolality level of less than about 275 mOsm/kg water, and has a lower concentration of fluid, sugars and salt than blood. Such a low osmolality helps, once ingested, to move solutions via osmosis across the gut walls and into the blood vessels, meaning a fast delivery occurs as the drink is rapidly absorbed across the gut lining. In particular, by having a high tonicity, the ability of an extracellular solution to move deliverables such as the water into or out of the cells of the human body by osmosis is also high.

Such decreases in osmolality is in direct contradiction to other types of systems such as isotonic drinks and hypertonic drinks. For example, isotonic drinks may have an osmolality ranging from about 275 to about 330 mOsm/kg water, and has a similar concentration of fluid, sugars, and salt than blood, leading to a slower absorption than hypotonic drinks. Finally, hypertonic drinks may have an osmolality between about 492 mOsm/kg water and about 784 mOsm/kg water, and has a higher concentration of fluids, sugars and salt than blood (e.g., juices, nectars, energizing drinks, and colas), leading to a slow absorption relative to the composition described herein.

Additionally, when the composition is introduced to the water, a number of processes begin with regards to the individual components. In an embodiment the primary carrier and the optional secondary carrier will both begin to absorb the water and the electrolytes. For example, the monosaccharides in the mix will begin to physically absorb the water and the electrolytes. Further, the polysaccharides in the mix will also begin to physically absorb the water and the electrolytes as well.

Once the drink is ready, the drink may be ingested by any suitable method, such as drinking. Once in the body, the monosaccharides make their way through the body and towards individual cells, where they immediately enter the cells. Additionally, as the monosaccharides enter the cells, the deliverables that are absorbed by the monosaccharides (e.g., the water and the electrolytes in embodiments where the deliverable is water) will also enter the cells, thereby bringing the desired deliverables (e.g., the absorbed water and the electrolytes) into the cell with it at a fast rate, thereby providing an immediate delivery (e.g., hydration) to those cells.

However, the polysaccharides present within the drink are not able to immediately enter the cell as easily as the monosaccharides. Rather, the body (with, e.g., the help of the vitamin B complexes), will begin to chemically break down the polysaccharides into monosaccharides. Once the polysaccharides have been broken down into the newly formed monosaccharides, the newly formed monosaccharides are now able to enter the cells, thereby carrying the desired deliverables (e.g., the water and the electrolytes) that were originally absorbed by the polysaccharides into the cells.

However, because of the time it takes for the polysaccharides to be broken down into the monosaccharides, the deliverables that had been originally absorbed by the polysaccharides are delayed from entering the cell. As such, the deliverables originally absorbed by the monosaccharides enter the cells first (thereby providing for an immediate delivery), while the deliverables originally absorbed by the polysaccharides are delayed from immediately entering the cells.

As such, by providing multiple paths for the deliverables to enter the cells, a longer, more time-released process can be utilized while still providing the benefits of an immediate, faster delivery process. In particular, in an embodiment to deliver water, by delaying a portion of the water until the disaccharides are broken down into monosaccharides, the portion of the water and the electrolytes originally absorbed by the polysaccharides are used to hydrate cells at a later time. As such, the overall hydration process can be continued for a longer time.

By utilizing the embodiments described herein as a hydration and delivery system, a composition may be achieved that offers a fast absorption rate and replenishes not just electrolytes but also other nutrients that are lost from dehydration. As such, the drink can help modify the osmolality of the body (e.g., a measure of the water and nutrients in the blood plasma of the body), so that the body is well hydrated, e.g., to an osmolality level of between about 275 mOsmol/kg and about 290 mOsmol/kg. For reference, the osmolality of water is between 20 and 120 mOsmol/kg. In preferred embodiments, the compositions according to aspects of the disclosure have an osmolality of about 20 mOsmol/kg to about 65 mOsmol/kg, more preferably about 35 to about 40 mOsmol/kg, most preferably about 35 mOsmol/kg, and a hydration drink containing the composition and electrolytes has an osmolality of about 48 mOsm/kg. Therefore, the hydration drink can hydrate the body as rapidly as water.

Of course, while a specific embodiment in which the delivery system is utilized to deliver water and other deliverables is described above, this is intended to be illustrative and is not intended to limit the embodiments to a hydration delivery system. Rather, the delivery system described could be used to deliver a wide variety of deliverables. For example, in another embodiment, the delivery system may be utilized to deliver a beauty product such as collagen to the cells of the human body. In still other embodiments, the delivery system may be utilized to deliver drugs (such as, for example and without limitation, ibuprofen, acetaminophen, naproxen, or other NSAIDS or any other drug to be contemplated) to the human body. Other embodiments relate to the use of the delivery system to deliver natural ingredients which may be selected and combined based on their specific properties and effects on the human body. All such embodiments may be utilized, and all such embodiments are fully intended to be included within the scope of the embodiments.

### EXAMPLES

The invention will now be described in connection with the following, non-limiting examples.

### Example 1: Preparation and Analysis of Composition Containing Potato Starch

A composition was prepared by combining the components shown in the following table in the listed amounts. The components (all in powder form) were weighed, added to a plow or V blender and mixed for 7 minutes. The blend was then tested to determine the osmolality using a Vapro model 5600; the osmolality was determined to be 38 mOsmol/kg H₂O.

| Component | Weight | |
|---|---|---|
| Non-GMO Potato Maltodextrin DE 9 | 3.50 | g |
| L-Glycine (Amino Acid) | 250 | mg |
| L-Alanine | 150 | mg |
| Vitamin C (Ascorbic Acid) | 92 | mg |
| Taurine | 250 | mg |
| Astragalus Root Powder | 50 | mg |
| Vitamin B2 | 50 | mg |
| Vitamin B6 | 50 | mg |
| Vitamin B3 | 16 | mg |
| Vitamin B12 | 125 | mcg |

### Example 2: Preparation and Analysis of Composition Containing Sweet Potato Starch

A composition was prepared by combining the components shown in the following table in the listed amounts. The components (all in powder form) were weighed, added to a plow or V blender and mixed for 7 minutes. The blend was then tested to determine the osmolality using a Vapro model 5600; the osmolality was determined to be 55 mOsmol/kg H₂O.

| Component | Weight | |
|---|---|---|
| Sweet Potato Starch | 3.50 | g |
| L-Glycine (Amino Acid) | 250 | mg |
| L-Alanine | 150 | mg |
| Vitamin C (Ascorbic Acid) | 92 | mg |
| Taurine | 250 | mg |
| Astragalus Root Powder | 50 | mg |
| Vitamin B2 | 50 | mg |
| Vitamin B6 | 50 | mg |
| Vitamin B3 | 16 | mg |
| Vitamin B12 | 125 | mcg |

### Example 3: Preparation and Analysis of Composition Containing Rice Starch

A composition was prepared by combining the components shown in the following table in the listed amounts. The components (all in powder form) were weighed, added to a plow or V blender and mixed for 7 minutes. The blend was then tested to determine the osmolality using a Vapro model 5600; the osmolality was determined to be 61 mOsmol/kg H₂O.

| Component | Weight | |
|---|---|---|
| Rice Starch | 3.50 | g |
| L-Glycine (Amino Acid) | 250 | mg |
| L-Alanine | 150 | mg |
| Vitamin C (Ascorbic Acid) | 92 | mg |
| Taurine | 250 | mg |
| Astragalus Root Powder | 50 | mg |
| Vitamin B2 | 50 | mg |
| Vitamin B6 | 50 | mg |
| Vitamin B3 | 16 | mg |
| Vitamin B12 | 125 | mcg |

### Example 4: Preparation and Analysis of Composition Containing Oat Starch

A composition was prepared by combining the components shown in the following table in the listed amounts. The components (all in powder form) were weighed, added to a plow or V blender and mixed for 7 minutes. The blend was then tested to determine the osmolality using a Vapro model 5600; the osmolality was determined to be 62 mOsmol/kg H₂O.

| Component | Weight | |
|---|---|---|
| Oat Starch | 3.50 | g |
| L-Glycine (Amino Acid) | 250 | mg |
| L-Alanine | 150 | mg |
| Vitamin C (Ascorbic Acid) | 92 | mg |
| Taurine | 250 | mg |
| Astragalus Root Powder | 50 | mg |
| Vitamin B2 | 50 | mg |
| Vitamin B6 | 50 | mg |
| Vitamin B3 | 16 | mg |
| Vitamin B12 | 125 | mcg |

### Example 5 (Prophetic): Preparation of Libido Enhancer

A libido enhancer is prepared by combining the ingredients shown in Table B below in the specified amounts by any method known in the art. In Table B, the “Delivery system” refers to a composition as described in any of Examples 1˜4 or an analogous composition containing an alternative resistant starch. All of the components shown in Table B are commercially available.

TABLE B
Libido Enhancer

| | Approximate |
|---|---|
| Component | Weight %* |
| Delivery system | 65% |
| Fenugreek Seed Extract | 15% |
| Horny Goat Weed Extract (Epimedium Grandiflorium) | 6% |
| Boron Citrate 5% | 6% |
| Tongkat Ali Extract | 3% |
| Caffeine | 3% |
| Zinc Picolinate (20% Zn) | 2% |

*total is greater than 100% due to rounding.

### Example 6 (Prophetic): Preparation of Hangover Recovery Composition

A hangover recovery composition was prepared by combining the ingredients shown in Table C below in the specified amounts by any method known in the art. In Table C, the “Delivery system” refers to a composition as described in any of Examples 1˜4 or an analogous composition containing an alternative resistant starch. All of the components shown in Table C are commercially available.

TABLE C
Hangover Recovery Composition

| | Approximate |
|---|---|
| Component | Weight %* |
| Delivery system | 43% |
| Niacinamide (B3) | 0.4% |
| Vitamin B6/Pyridoxine HCL | 1% |
| Vitamin B12 - 1% (Cyanocobalamin) | 1% |
| Caffeine Anhydrous | 4% |
| Siberian Ginseng | 2% |
| Sea Salt | 4% |
| L-Glutamine | 19% |
| Vine Tea Extract 98% Dihydromyricetin | 4% |
| Magnesium Bisglycinate Chelate Powder 10% Mg | 5% |
| Vitamin B5 | 2% |
| Potassium Citrate | 8% |
| Trisodium Citrate Dihydrate | 7% |
| Calcium Bisglycinate Chelate Powder 18% Ca | 1% |

*total is greater than 100% due to rounding.

### Example 7 (Prophetic): Preparation of Energy/Focus Booster Composition

An energy/focus booster composition is prepared by combining the ingredients shown in Table D below in the amounts shown by any method known in the art. In Table D, the “Delivery system” refers to a composition as described in any of Examples 1˜4 or an analogous composition containing an alternative resistant starch. All of the components shown in Table D are commercially available.

TABLE D
Energy/Focus Booster Composition

| Component | Approximate Weight % |
|---|---|
| Delivery system | 66% |
| Choline L-Bitartrate | 3% |
| Lions Mane Mushroom Extract | 14% |
| Ashwagandha Root Extract | 9% |
| Caffeine Anhydrous | 5% |
| SunTheanine | 3% |
| Saffractive Saffrom | 0.5% |

### Example 8 (Prophetic): Preparation of Immune Boost Composition

An immune boost composition is prepared by combining the ingredients shown in Table E below in desired amounts by any method known in the art. In Table E, the “Delivery system” refers to a composition as described in any of Examples 1˜4 or an analogous composition containing an alternative resistant starch. All of the components shown in Table E are commercially available.

TABLE E
Immune Boost Composition

| Component | Approximate Weight % |
|---|---|
| Delivery system | 50% |
| Elderberry Extract | 44% |
| Vitamin C (Ascorbic Acid) | 5% |
| Vitamin D3 (0.25%) (Cholecalciferol) | 1% |

### Example 9 (Prophetic): Preparation of Stress Relief Composition

A stress relief composition is prepared by combining the ingredients shown in Table F below in desired amounts by any method known in the art. In Table F, the “Delivery system” refers to a composition as described in any of Examples 1˜4 or an analogous composition containing an alternative resistant starch. All of the components shown in Table F are commercially available.

TABLE F
Stress Relief Composition

| Component | Approximate Weight %* |
|---|---|
| Delivery system | 56% |
| Turmeric Extract (95% Curcuminoids) | 12% |
| Black Pepper (95% Piperine) | 0.5% |
| Ashwagandha Root Extract | 15% |
| Valerian Root Extract | 10% |
| Lavender Extract | 7% |

*total is greater than 100% due to rounding.

### Example 9 (Prophetic): Preparation of Weight Control Composition

A weight control composition is prepared by combining the ingredients shown in Table G below in desired amounts by any method known in the art. In Table G, the “Delivery system” refers to a composition as described in any of Examples 1˜4 or an analogous composition containing an alternative resistant starch. All of the components shown in Table G are commercially available.

TABLE G
Weight Control Composition

| Component | Approximate Weight %* |
|---|---|
| Delivery system | 54% |
| Turmeric | 23% |
| Acetyl L-Carnitine | 6% |
| Conjugated linoleic acid (CLA) | 6% |
| Lions Mane Mushroom Extract | 12% |

*total is greater than 100% due to rounding.

It will be appreciated by those skilled in the art that changes could be made to the embodiments described above without departing from the broad inventive concept thereof. It is understood, therefore, that this invention is not limited to the particular embodiments disclosed but it is intended to cover modifications within the spirit and scope of the present invention as defined by the appended claims.

We claim:

1. A delivery composition comprising at least one carrier, wherein the at least one carrier comprises a resistant starch selected from the group consisting of potato, sweet potato, rice, oats, pasta, and a lentil.

2. The delivery composition according to claim 1, wherein the resistant starch is potato maltodextrin.

3. The delivery composition according to claim 2, wherein the potato maltodextrin is potato maltodextrin DE 9.

4. The delivery composition according to claim 1, wherein the resistant starch is a potato starch, a sweet potato starch, a rice starch, or an oat starch.

5. The delivery composition according to claim 1, further comprising at least one amino acid.

6. The delivery composition according to claim 5, wherein the delivery composition comprises L-glycine and alanine.

7. The delivery composition according to claim 1, further comprising at least one vitamin.

8. The delivery composition according to claim 7, wherein the delivery composition comprises at least one of vitamin C, vitamin B3, vitamin B6, vitamin B2, and vitamin B12.

9. The delivery composition according to claim 1, further comprising at least one component selected from the group consisting of taurine and astragalus root powder.

10. The delivery composition according to claim 1, further comprising L-glycine, L-alanine, vitamin C, vitamin B3, vitamin B2, vitamin B6, taurine, and astragalus root powder.

11. The delivery composition according to claim 10, wherein the resistant starch is present in an amount of about 75 to about 85 weight %, the L-glycine is present in an amount of about 5 to about 7 weight %, the L-alanine is present in an amount of about 3 to about 4 weight %, the vitamin C is present in an amount of about 1 to about 3 weight %, the vitamin B6 is present in an amount of about 0.5 to about 2 weight %, the vitamin B3 is present in an amount of about 0.1 to about 0.7 weight %, the vitamin B2 is present in an amount of about 0.5 to about 2 weight %, the taurine is present in an amount of about 5 to about 7 weight %, and the astragalus root powder is present in an amount of about 0.5 to about 2 weight %, based on a total weight of the delivery composition being 100 percent.

12. The delivery composition according to claim 1, wherein the composition has an osmolality of about 20 to about 65 mOsmol/kg.

13. A hydration drink comprising the delivery composition according to claim 1, water, and at least one electrolyte.

14. The hydration drink according to claim 13, wherein the osmolality is about 48 mOsmol/kg.

15. A drug product comprising the delivery composition according to claim 1 and at least one active ingredient.
